(19) United States
(12) Patent Application Publication
(10) Pub. No.: US 2012/0077234 A1
(43) Pub. Date: Mar. 29, 2012

(54) METHOD AND SYSTEM FOR MICROBIAL CONVERSION OF CELLULOSE TO FUEL

(76) Inventor: David A. Hazlebeck, El Cajon, CA (US)

(21) Appl. No.: 12/893,281

(22) Filed: Sep. 29, 2010

(51) Int. Cl. C12P 7/64; C10G 3/00; C10L 1/18; C12M 1/00; C10L 1/19

(52) U.S. Cl. 435/134; 435/289.1; 44/388; 44/350; 44/403; 44/389; 585/242

(57) ABSTRACT

A method and system are provided for producing biofuel from cellulosic feedstock. In the method, the cellulosic feedstock is pretreated to separate cellulose, hemicellulose, and lignin. Thereafter, the cellulose and hemicellulose are converted into sugars through enzymatic hydrolysis. Then, the sugars are converted into lipids, e.g. triglycerides, through a microbial process. Specifically, heterotrophic microalgae is grown on the triglycerides and forms triglycerides. While triglycerides are formed from the cellulose and hemicellulose, the lignin is converted into ringed hydrocarbons, such as aromatic hydrocarbons and cycloalkanes, e.g., cycloparaffins. To form the biofuel, the triglycerides and ringed hydrocarbons are processed together. During this step, the triglycerides are converted into straight chain paraffins and esters. Preferably, the biofuel is a surrogate for jet quality JP-8 fuel.

### FIELD OF THE INVENTION

The present invention pertains generally to processes for producing biofuel. More particularly, the present invention pertains to methods for processing cellulosic material to convert its constituents into biofuel in an optimal manner. The present invention is particularly, but not exclusively, useful as a system and method for producing biofuel from hydrocarbons derived from cellulose and lignins supplied from a biomass feedstock.

### BACKGROUND OF THE INVENTION

As worldwide petroleum deposits decrease, there is rising concern over shortages and the costs that are associated with the production of hydrocarbon products. As a result, alternatives to products that are currently processed from petroleum are being investigated. In this effort, biofuels have been identified as a possible alternative to petroleum-based transportation fuels. In general, biofuels are comprised of mono-alkyl esters of long chain fatty acids derived from plant oils or animal fats. In industrial practice, biofuel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by minimizing the costs of the plant source and by maximizing the energy extracted from the plant source. For the former concern, the use of cellulosic materials such as agricultural wastes, paper and food wastes, and quick-growing energy crops can reduce feedstock costs. Specifically, the cellulosic raw material is plentiful as it is present in every plant and plant-derived product. It is estimated that between 500 million and 1 billion tons of cellulosic materials are discarded in the United States each year. This includes over 30 million dry tons of urban wood wastes, 90 million dry tons of primary mill residues, 45 million dry tons of forest residues, and 150 million dry tons of corn stover and wheat straw. Due to its ubiquity, the cost of cellulosic material for use as biofuel feedstock is estimated to be about $30 to $60 per ton. In addition to using inexpensive raw materials, biofuel production processes that utilize cellulosic waste may substantially reduce the rate of landfill use. For instance, paper, cardboard, and packaging comprise about 40% of the solid waste sent to landfills in the United States each day.

In order to make use of these cellulosic materials as feedstock in biofuel production, their processing into fuel must be optimized. Therefore, a biofuel production method should utilize each possible source of energy within the cellulosic feedstock in order to efficiently produce a biofuel.

In light of the above, it is an object of the present invention to provide a system and method for producing biofuel which maximizes the energy provided from a biomass feedstock. Another object of the present invention is to provide a system and method for producing a jet fuel surrogate biofuel produced from the enzymatic conversion of cellulosic material into sugars. Still another object of the present invention is to provide a system and method for producing biofuel from the microbial conversion of sugars to lipids. Still another object of the present invention is to provide a method and system for producing biofuel that converts lignin in a biomass feedstock into hydrocarbons for use in the biofuel. Another object of the present invention is to provide a method and system for producing biofuel that processes hydrocarbons derived from cellulosic materials along with hydrocarbons derived from lignin. Yet another object of the present invention is to provide a system and method for producing biofuel that is simple to implement, easy to use, and comparatively cost effective.

### SUMMARY OF THE INVENTION

In accordance with the present invention, a method and system are provided for producing biofuel from cellulosic materials. During the method, sugars are converted into triglycerides, rather than to the shorter chain alcohols commonly created during the formation of cellulosic ethanol and butanol. Importantly, triglycerides are much closer to traditional petroleum based fuels in energy content. In addition, unlike typical systems for converting cellulose into fuel, the present method utilizes algae fermentation of sugars. As a result, unlike the typical systems, large sterile reactors are not necessary. Moreover, the present method utilizes the lignin present in cellulosic feedstock to supply cyclic compounds, thereby reducing the amount of refining needed for the cellulose-derived hydrocarbons. As a result, the cellulose is converted into primarily straight chain paraffins or esters in a less-energy intensive process. Also, the use of lignin greatly increases the fuel energy per pound of cellulosic feedstock.

In operation, cellulosic feedstock is pretreated to separate the cellulose, hemicellulose, lignin, extractables, and co-products. Thereafter, the cellulose and hemicellulose are transformed into straight-chain hydrocarbons while the lignin is transformed into ringed hydrocarbons. Specifically, the cellulose and hemicellulose are first converted into sugars through hydrolysis. Then, the sugars are microbially converted into lipids by heterotrophic microalgae. After the lipids are extracted from the microalgae, they are converted into straight chain paraffins and esters.

Parallel to the processing of the cellulose and hemicellulose, the lignin is converted into hydrocarbons. Specifically, the lignin is converted into ringed hydrocarbons such as aromatic hydrocarbons and cycloalkanes like cycloparaffins. During the formation of the biofuel, the hydrocarbons derived from cellulosic material and the ringed hydrocarbons derived from lignin are blended and processed. Also, aromatic extractables separated at the pretreatment stage are added to the other hydrocarbons. As a result, a biofuel having a high energy content is produced to perform as a surrogate for jet fuel.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an operational flow chart illustrating the biofuel production method in accordance with the present invention;

FIG. 2 is an operational flow chart illustrating an embodiment of the initial treatment process identified in FIG. 1; and

FIG. 3 is a simplified process flow chart and energy balance for an embodiment of the methods of FIGS. 1 and 2 when using corn stover as the cellulosic feedstock.

### DESCRIPTION OF THE PREFERRED EMBODIMENTS

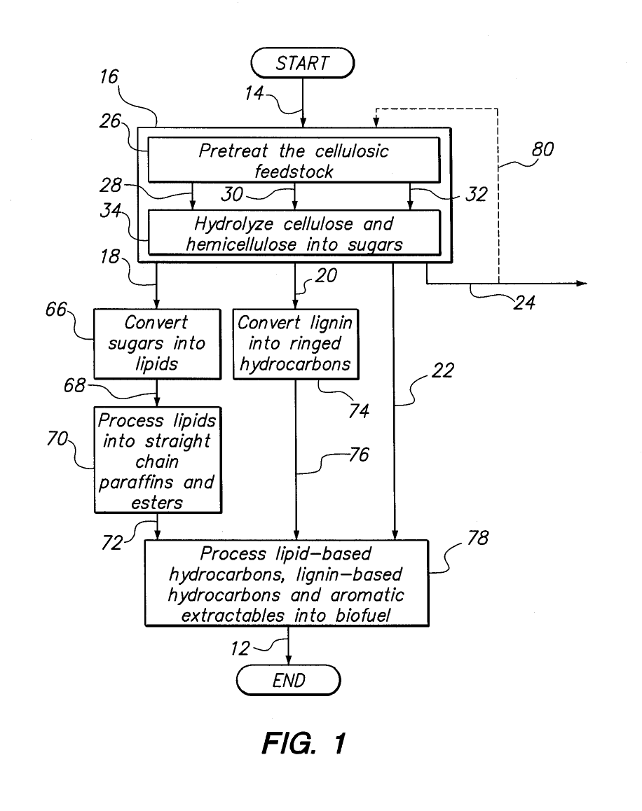

Referring initially to FIG. 1, a method for producing biofuel (indicated by arrow 12) from cellulosic biomass feedstock (arrow 14) is illustrated. Importantly, the method optimizes the use of inputs and reduces the losses of materials and energy. Generally, the feedstock 14 is comprised of biomass from a single species or from a variety of plant life. For instance, the feedstock 14 may include agricultural residues, such as corn stover, saw dust, rice hulls, distiller grains, straws, bagasse, cotton gin trash, palm oil wastes, etc. Also, the feedstock 14 may comprise energy crops grown specifically for their biomass, such as grasses, sweet sorghum, fast growing trees, etc. Further, the feedstock 14 may comprise waste products of paper, such as recycled newspaper, paper mill sludges, pulp mill waste, sorted municipal solid waste, etc., and/or green wastes, such as leaves, grass clippings, shrimp wastes, vegetable and fruit wastes, etc.

As shown in FIG. 1, the method commences with an initial treatment (at action block 16) of the feedstock 14. During this initial treatment 16, the feedstock 14 is converted into sugars (arrow 18), lignin (arrow 20), extractables (arrow 22), and co-products (arrow 24). As shown in FIG. 1, the initial treatment 16 may be comprised of two separate steps. Specifically, in FIG. 1, the feedstock 14 is first pretreated at action block 26 to enable later processing. Structurally, the cell walls of the biomass in the feedstock 14 form recalcitrant barriers that resist degradation and complicate processing of the biomass. Therefore, the pretreatment step 26 serves to physically and/or chemically break the cell walls of the biomass to facilitate further processing. As a result of the pretreatment step 26, cellulose (arrow 28) and hemicellulose (arrow 30) are released from the cellular barriers in the biomass. While the cellulose 28 and hemicellulose 30 are illustrated as separate streams from the non-cellulosic material (arrow 32), these components may be mixed after the pretreatment step 26.

Still referring to FIG. 1, it may be seen that the cellulose 28 and hemicellulose 30 undergo cellulose hydrolysis (cellulolysis) at action block 34. Specifically, the cellulose 28 and hemicelluloses 30 molecules are composed of long chains of sugar molecules of various kinds. In the hydrolysis process, these chains are broken down to free the sugars 18 therefrom. Currently, there are two major cellulose hydrolysis processes: a chemical reaction using acids, and an enzymatic reaction. In the present invention, either method may be employed. If the latter is utilized, enzymes such as cellulase, xylanase, hemicellulase and/or other similar enzymes break down the cellulosic molecules into sugars 18. Due to the heterogeneity of biomass cell walls, multiple enzyme systems may be required for degradation. For example, cellulytic organisms generally secrete a variety of endo-acting glucanases, exo-acting cellubiohydrolases, glucosidases, and phosphorylases. The complexity and sheer number of different bonds found in the non-cellulosic components of biomass cell walls will require other enzyme systems that rival cellulose systems in their complexity. In the present system, improved enzyme systems may be used to improve the speed and performance of the conversion of cellulosic material 28, 30 to sugars 18, and to reduce the expense of converting cellulosic material 28, 30 to sugars 18.

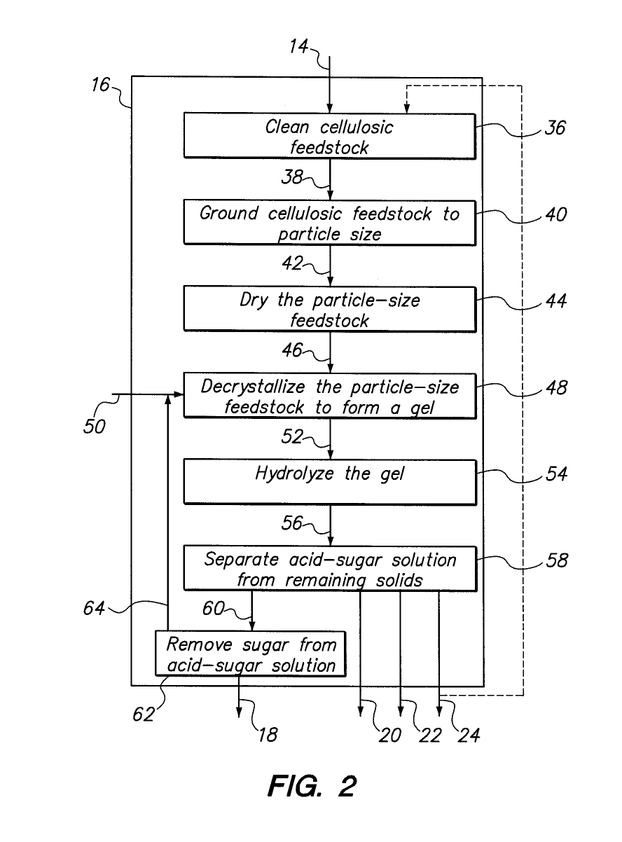

Depending on the biomass used in the feedstock 14 and upon other considerations, the initial treatment 16 of the feedstock 14 may involve strong acid hydrolysis, solvent extraction, screw extraction with a weak acid, steam explosion, microwave treatment, ammonia fiber expansion, alkaline wet oxidation and/or ozone pretreatment. Referring to FIG. 2, the process of strong acid hydrolysis is illustrated for exemplary purposes.

As shown in FIG. 2, for the strong acid hydrolysis process, the feedstock 14 is first cleaned at action block 36 to remove dirt or other foreign material. Then, the cleaned feedstock (arrow 38) is ground to particle size at action block 40 for use with the process equipment. Then, the ground material (arrow 42) is dried to a moisture content that is consistent with the acid concentration requirements for decrystallization (action block 44). Thereafter, the dried particle size feedstock (arrow 46) is decrystallized at action block 48. Specifically, an acid (arrow 50), such as sulfuric, hydrochloric, hydrofluoric, or phosphoric acid, is mixed with the feedstock 46 in a reaction chamber (not shown). As a result of the decrystallization step 48, a gel (arrow 52) is formed. As shown in FIG. 2, the gel 52 is hydrolyzed at action block 54 to produce a hydrolysate stream (arrow 56) including sugars 18 such as hexoses and pentoses in an acid-sugar solution. Thereafter, insoluble materials (lignin 20, extractables 22, co-products 24) are separated from the hydrolysate stream 56 by filtering and pressing at action block 58. Then, the remaining acid-sugar solution (arrow 60) is separated into its acid and sugar components at action block 62. Specifically, ion exchange resins separate these components without diluting the sugars 18. The acid (arrow 64) that is separated can be recirculated and reconcentrated for use in the decrystallization and hydrolysis steps as shown.

While a strong acid hydrolysis method is illustrated in FIG. 2, as noted above, other initial treatments 16 may be used. When solvent extraction is used as the initial treatment 16, two principle steps are involved. First, a modified solvent extraction stage treats the feedstock 14 with aqueous ethanol at elevated temperatures. As a result, much of the large lignin polymers in the feedstock 14 are converted into smaller molecular weight fragments. These fragments dissolve in the hot ethanol-based liquor. While these fragments retain most of the lignin's chemical structure and properties, sulfur is not introduced into their chemical structure as in other solvent extraction methods. Therefore, the lignin retains its desirability. Further, during the solvent extraction stage, acetyl groups on the hemicellulose are hydrolyzed to form acetic acid, while most of the hemicelluloses polysaccharide structure is hydrolyzed to a mixture of simple sugars and small chain oligosaccharides. Additionally, most of the lipophylic components of the feedstock 14, generally called extractables 22, dissolve in the hot liquor. The products of the first stage, therefore, are a cellulose-rich fiber and aqueous ethanol liquor that contains the dissolved extracted materials. The liquor is then treated in a series of unit processes to recover ethanol, furfural (a valuable extractables fraction), a second lignin fraction, hexose sugars derived from the hemicelluloses, and acetic acid. In the second stage, a cellulose enzyme complex converts the cellulose-rich fiber to sugars.

In another embodiment, the initial treatment 16 involves screw extraction with a weak acid. In this step, the feedstock 14 is first ground into particles and then fed to a pressurized chamber designed for counter-current processing. Next, a biomass fractionation process separates the three primary constituents of the feedstock 14: cellulose 28, hemicellulose 30, and lignin 20. This continuous fractionation process employs a counter-current extraction technique that separates the cellulose 28 and hemicellulose 30 fractions from the lignin 20 fractions into two high-quality liquid streams. As a result, one stream contains a solid fraction with relatively pure cellulose fiber. Thereafter, the cellulose is converted to sugar as discussed above.

In still another embodiment, steam explosion is used in the initial treatment 16. In this embodiment, the feedstock 14 is first prepared by properly sizing fibers in the feedstock 14 and removing dirt and ash. During this process, the surface area of the fibers is increased for maximum exposure during bioprocessing. Thereafter, the biomass enters a high-pressure continuous feeder where heat and moisture are added before a rapid depressurization. This step is known as steam explosion. After the steam explosion step, the materials are separated in a cyclone. Afterwards, the cellulose and hemicellulose go through enzymatic hydrolysis into C6 and C5 sugars. Further, the lignin fraction is taken off for further processing. The steam explosion process avoids the use of costly acids or recovery systems, and can provide significant cost reductions.

In yet another embodiment, the initial treatment 16 involves the use of microwave energy. Specifically, for feedstock 14 including wood material, the wood or wood bark is pretreated with microwave energy to open it up to acid, basic, or alkaline peroxide solutions. Thereafter, the cellulosic content of the wood is hydrolyzed with commercial enzymes to sugars. Further, pulping liquors can be used to separate carbohydrates and lignin, which can be used in further processing.

Referring back to FIG. 1, the further processing of the sugars 18 after the initial treatment 16 is illustrated. At action block 66, the method provides for microbial conversion of the sugars 18 into lipids 68. Specifically, the sugars 18 are fed to heterotrophic microalgae that biochemically convert sugars 18 to triglycerides 68, other acylglycerols, or other esters. In action block 66, a variety of oleaginous microalgae may efficiently grow on the sugars 18 derived from cellulose 28 and hemicellulose 30. Further, these microalgae may be triggered to optimize lipid production, and to produce a desired strain of lipids 68. Specifically, microalgae may be selected to produce a profile of lipids 68 from short-chain saturated fatty acids to long-chain polyunsaturated fatty acids. During action block 66, the lipids 68 are extracted from the microalgae, preferably through traditional organic solvent extraction methods, including aqueous extraction techniques. While not illustrated, co-products 24 may also be fed to the microalgae to facilitate growth and sugar conversion to lipids 68. After the lipids 68 are extracted, they are processed at action block 70 into hydrocarbons 72 such as straight chain paraffins and esters.

As shown in FIG. 1, the lignin 20 isolated by the initial treatment 16 is converted at action block 74 into ringed hydrocarbons 76. Specifically, the lignin 20 is converted into aromatic hydrocarbons and cycloalkanes, such as cycloparaffin compounds, 76, according to known methods. Thereafter, the hydrocarbons 72 produced from lipids 68, and the hydrocarbons 76 produced from lignins 20 are processed into biofuel 12 at action block 78. Further aromatic extractables 22 may also be used to produce biofuel 12 at action block 78. For the present invention, the biofuel 12 is preferably a surrogate for JP-8, a jet propellant fuel.

Further, some of the co-products 24 can be recycled and used during steps within the process to optimize efficiency. For instance, acetic acid can be used as feedstock (as shown by arrow 80). Also, oil elicitor can be used in action block 66 during microbial lipid 68 production. Further, furfural and certain components of the extractables 22 can be sold. In addition to co-products 24 from the initial treatment 16, secondary co-products, such as animal feed, can also be obtained from the remaining algae after extraction of the lipids 68.

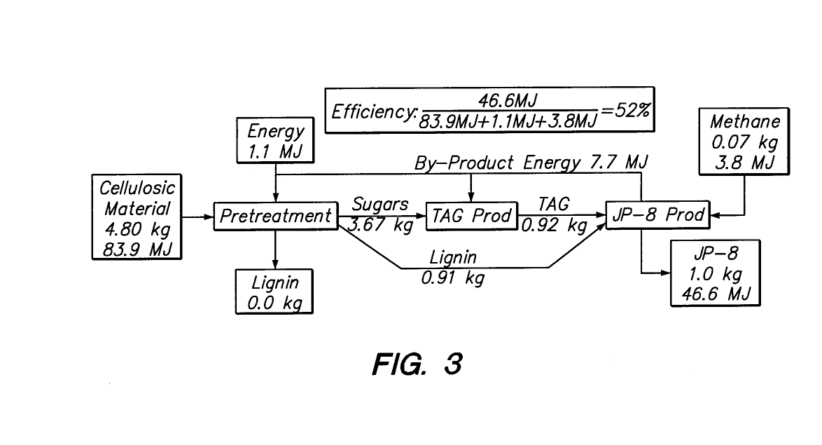

Referring to FIG. 3, a simplified process flow diagram and energy balance is illustrated for the methods disclosed in FIGS. 1 and 2 when used with corn stover as the feedstock 14. As shown, the efficiency of the method is 52% and 1 kg of JP-8 surrogate biofuel 12 is produced from 4.80 kg of feedstock 14.

While the particular Method and System for Microbial Conversion of Cellulose to Fuel as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for producing biofuel from cellulosic feedstock comprising the steps of:
converting cellulose from the feedstock into lipids;
converting lignin from the feedstock into ringed hydrocarbons; and
processing the lipids and ringed hydrocarbons into biofuel.

2. A method as recited in claim 1 further comprising the steps of:
pretreating the cellulosic feedstock to separate the cellulose and the lignin therefrom;
converting the cellulose into sugars; and
converting the sugars into lipids.

3. A method as recited in claim 2 wherein the ringed hydrocarbons comprise aromatic hydrocarbons and cycloalkanes.

4. A method as recited in claim 2 wherein, during the processing step, the lipids are converted into straight chain paraffins and esters.

5. A method as recited in claim 2 wherein the pretreating step further separates hemicellulose and aromatic extractables from the cellulosic feedstock, wherein the hemicellulose is converted into the sugars with the cellulose, and wherein the aromatic extractables are processed into the biofuel with the ringed hydrocarbons and the lipids.

6. A method as recited in claim 5 wherein the pretreating step further separates co-products from the cellulosic feedstock, wherein the co-products are selected from the group consisting of acetic acid, oil elicitor, and furfural.

7. A method as recited in claim 2 wherein the cellulose is converted into the sugars through enzymatic hydrolysis.

8. A method as recited in claim 7 wherein the sugars are converted into lipids through a microbial process.

9. A method as recited in claim 8 wherein the sugars are converted into lipids by heterotrophic microalgae.

10. A method for producing biofuel from cellulosic feedstock comprising the steps of:
pretreating the cellulosic feedstock to facilitate further treatment of cellulose and lignin therein;
transforming the cellulose into lipids;
transforming the lignin into ringed hydrocarbons; and
processing the lipids and ringed hydrocarbons into biofuel.

11. A method as recited in claim 10 wherein the lipids comprise triglyceride.

12. A method as recited in claim 11 wherein, during the processing step, the lipids are converted into straight chain paraffins and esters.

13. A method as recited in claim 12 wherein the ringed hydrocarbons comprise aromatic hydrocarbons and cycloalkanes.

14. A method as recited in claim 10 wherein the cellulose is transformed into lipids by converting the cellulose into sugars through enzymatic hydrolysis, and by thereafter converting the sugars into lipids through a microbial process.

15. A method as recited in claim 14 wherein the sugars are converted into lipids by heterotrophic microalgae.

16. A system for producing biofuel from cellulosic feedstock comprising:
means for pretreating the cellulosic feedstock to facilitate further treatment of cellulose and lignin therein;
first means for converting the cellulose into sugars;
second means for converting the sugars into lipids;
third means for converting the lignin into ringed hydrocarbons; and
means for processing the lipids and ringed hydrocarbons into biofuel.

17. A system as recited in claim 16 wherein the lipids comprise triglyceride, and wherein the ringed hydrocarbons comprise aromatic hydrocarbons and cycloalkanes.

18. A system as recited in claim 17 wherein the processing means converts the lipids into straight chain paraffins and esters.

19. A system as recited in claim 18 wherein the first means converts the cellulose into the sugars through enzymatic hydrolysis, and wherein the second means converts the sugars into lipids through a microbial process.

20. A system as recited in claim 19 wherein the second means comprises heterotrophic microalgae.
